(19) United States
(12) Patent Application Publication
(10) Pub. No.: US 2022/0105226 A1
(43) Pub. Date: Apr. 7, 2022

(54) MINI UVC AIR STERILIZER

(71) Applicant: John Technology Holdings Limited, Kowloon (HK)

(72) Inventor: Se Kit Yuen, Kowloon (HK)

(73) Assignee: John Technology Holdings Limited, Kowloon (HK)

(21) Appl. No.: 17/131,628

(22) Filed: Dec. 22, 2020

(30) Foreign Application Priority Data
Oct. 5, 2020 (HK) 32020017231.2

(51) Int. Cl. A61L 9/20; A61L 9/22

(52) U.S. Cl. CPC A61L 9/20; A61L 9/22; A61L 2209/11; A61L 2209/15; A61L 2209/14; A61L 2209/12

(57) ABSTRACT

The application discloses an air sterilizer. The air sterilizer includes a housing an ultraviolet light generator, and a reflector. The housing is provided with a first cavity, and the housing is provided with an air inlet and an air outlet, both of which are located at the first cavity. The ultraviolet light generator is also located in the first cavity. In the embodiment of the present application, an ultraviolet light generator is placed in the first cavity of the air sterilizer. The air flows in through the air inlet. When the air flows through the first cavity, it is sterilized by the ultraviolet light generator, and then flows out from the air outlet. The toxic or harmful substances in the air can be inactivated by ultraviolet light sterilization, which improves the sterilization and disinfection ability of the air sterilizer, and solves the problem of poor sterilization and disinfection effects of air sterilizers in the prior art.

### CROSS-REFERENCE TO RELATED APPLICATIONS

This application claims priority to Hong Kong Short-Term Patent Application No. 32020017231.2 filed Oct. 5, 2020, the entirety of which is incorporated herein by reference.

### FIELD OF THE INVENTION

This application relates to the field of air sterilization, in particular to an air sterilizer.

### BACKGROUND OF THE INVENTION

In the prior art, air sterilizers usually use activated carbon filters to adsorb toxic or harmful substances in the air, and the adsorbed toxic or harmful substances are easily separated to form secondary pollution. That is, the air sterilizers in the prior art have the problem of poor sterilization effect.

### SUMMARY OF THE INVENTION

The embodiments of the present application provide an air sterilizer, which solves the problem of poor sterilization and disinfection effects of air sterilizers in the prior art.

In order to achieve the above objective, an embodiment of the present application provides an air sterilizer. The air sterilizer includes a housing and an ultraviolet light generator. The housing is provided with a first cavity, an air inlet and an air outlet. Such air inlet and air outlet are located at the first cavity. The ultraviolet light generator is also located in the first cavity.

Optionally, the air sterilizer further includes a reflector located in the first cavity. The reflector is arranged corresponding to the ultraviolet light generator, such that the reflecting surface of the reflector faces the ultraviolet light generator.

Optionally, the air sterilizer further includes a fan, which fan is located in the first cavity, and placed between the ultraviolet light generator and the air outlet.

Optionally, the air sterilizer further includes two hanging loops for strap, and such hanging loops are respectively connected to opposite sides of the housing.

Optionally, the air sterilizer further includes a negative ion generator and a cathodic high-voltage carbonized fiber. A second cavity is also provided in the housing, and the negative ion generator is located in the second cavity. The first end of the cathodic high-voltage carbonized fiber is connected to the negative ion generator, while the second end of the cathodic high-voltage carbonized fiber passes through the second cavity and the first cavity in turn and located at the air outlet.

Optionally, the air sterilizer further includes a filter. The filter is located in the first cavity, and placed between the ultraviolet light generator and the air inlet.

Optionally, the air sterilizer further includes a power interface arranged on the outside of the housing and a rechargeable battery arranged in the second cavity. The power interface, the ultraviolet light generator and the negative ion generator are all electrically connected to the rechargeable battery.

Optionally, the air sterilizer further includes a control device and indicator lights located in the second cavity. The housing is provided with openings corresponding to the indicator lights. The control device is electrically connected with the ultraviolet light generator, the negative ion generator and the indicator lights respectively.

Optionally, the air sterilizer further includes a first sub reflector, a second sub reflector, and a third sub reflector, which sub reflectors are sequentially connected, such that the second sub reflector is located between the first sub reflector and the third sub reflector.

Optionally, the air sterilizer further includes movable shutters arranged at the air outlet.

In the embodiment of the present application, the air sterilizer includes a housing and an ultraviolet light generator. The housing is provided with a first cavity, an air inlet and an air outlet. Such air inlet and air outlet are located at the first cavity. The ultraviolet light generator is also located in the first cavity. Specifically, an ultraviolet light generator that generates short-wave ultraviolet light can be selected, such as UVC (UVC being short-wave ultraviolet light) lamp beads. The function of sterilization is achieved by the short-wave ultraviolet light destroying the molecular structure of DNA (deoxyribonucleic acid) or RNA (ribonucleic acid) in cells of microorganisms, causing death of their growth cells and/or regenerative cells. In the embodiment of the present application, an ultraviolet light generator is placed in the first cavity of the air sterilizer. The air flows in through the air inlet. When the air flows through the first cavity, it is sterilized by the ultraviolet light generator, and then flows out from the air outlet. The toxic or harmful substances in the air can be inactivated by ultraviolet light sterilization, which improves the sterilization and disinfection ability of the air sterilizer, and solves the problem of poor sterilization and disinfection effects of existing air sterilizers.

### DESCRIPTION OF THE DRAWINGS

In order to more clearly describe the technical solutions in the embodiments of the present invention, the following are descriptions of the accompanying drawings. Obviously, the following drawings are only embodiments of the present invention. For those of ordinary skill in the art, without any creative work they may produce other drawings based on the following drawings.

FIG. 1 is a front view of an air sterilizer provided by an embodiment of the present invention;

FIG. 2 is a left side view of an air sterilizer provided by an embodiment of the present invention;

FIG. 3 is a right side view of the air sterilizer provided by the embodiment of the present invention;

FIG. 4 is a rear view of the air sterilizer provided by the embodiment of the present invention;

FIG. 5 is a top view of an air sterilizer provided by an embodiment of the present invention;

FIG. 6 is a bottom view of an air sterilizer provided by an embodiment of the present invention;

FIG. 7 is the first perspective of an air sterilizer provided by an embodiment of the present invention;

FIG. 8 is the second perspective of an air sterilizer provided by an embodiment of the present invention;

FIG. 9 is the first vertical cross-sectional view of an air sterilizer provided by an embodiment of the present invention;

FIG. 10 is the second vertical cross-sectional view of an air sterilizer provided by an embodiment of the present invention;

FIG. 11 is a horizontal cross-sectional view of an air sterilizer provided by an embodiment of the present invention;

FIG. 12 is an exploded perspective view of an air sterilizer provided by an embodiment of the present invention.

### DESCRIPTION OF AN EMBODIMENT

The technical solutions in the embodiments of the present invention will be clearly and completely described below in conjunction with the accompanying drawings. Obviously, the described embodiments are only a part of the embodiments of the present invention, not all the embodiments. On the basis of the embodiments of the present invention, all other embodiments obtained by those of ordinary skill in the art without creative work shall fall within the protection scope of the present invention.

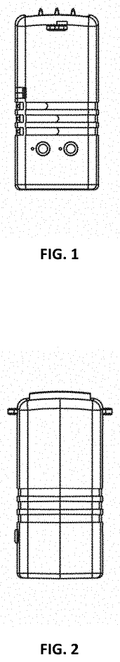

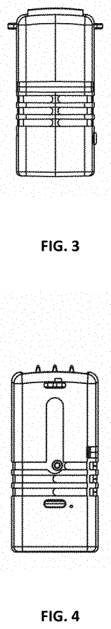

Referring to FIG. 1, the air sterilizer provided by the embodiment of the present application includes a housing and an ultraviolet light generator 23. The housing is provided with a first cavity 21. The housing is provided with an air inlet 4 and an air outlet 2. The ultraviolet light generator 23, the air inlet 4 and the air outlet 2 are all located in the first cavity 21.

Specifically, the material of the housing of the air sterilizer provided in the embodiments of the present application may be a plastic material or an aluminum alloy material. The housing may be made of the first half-shell 1 and the second half-shell 6 to be fixedly connected by fasteners such as screws. The housing formed by the two half-shells can be in the shape of a rectangular cuboid for easy placement on the ground. When the housing is in the shape of a rectangular cuboid, the top surface of the housing is provided with an air outlet 2 and there may be an air inlet 4 on one of the four sides of the housing. Further or alternatively, an air inlet 4 may be provided on each of the four sides so as to increase the air intake and improve the efficiency of air sterilization.

The internal components of the first cavity 21, such as the ultraviolet light generator 23, can be fixed on the housing by bolting or magnetic attraction. The bolting can be a bolt-nut connection, the bolt of which can be welded to the housing. The ultraviolet light generator 23 is provided with a nut. One end of the bolt is welded to the inner wall surface of the housing, and the other end passes through the nut on the ultraviolet light generator 23 which can be fixed in the first cavity 21 through tightening of the nut. For magnetic attraction, a strong magnet can be welded on the inner wall of the housing, and a metal plate can be soldered to the ultraviolet light generator 23, such that the ultraviolet light generator 23 can be fixed in the first cavity 21 through the adsorption effect of the metal by the magnet.

When the air sterilizer is in operation, the air flows in through the air inlet 4, passes through the ultraviolet light generator in the first cavity 21, and flows out from the air outlet 2. The function of sterilization or disinfection is achieved by the short-wave ultraviolet light destroying the molecular structure of DNA (deoxyribonucleic acid) or RNA (ribonucleic acid) in cells of microorganisms, causing death of their growth cells and/or regenerative cells. Therefore, the short-wave ultraviolet light generated by the ultraviolet light generator 23 has a sterilization and disinfection effect on the air flowing through the first cavity 21.

In the embodiment of the present application, the air sterilizer includes a housing and an ultraviolet light generator 23. The housing is provided with a first cavity 21, an air inlet 4 and an air outlet 2. The ultraviolet light generator is located in the first cavity 21. In the embodiment of the present application, an ultraviolet light generator 23 emitting short-wave ultraviolet light when in operation is placed in the first cavity 21 of the air sterilizer. The air flows in through the air inlet 4, passes through the first cavity 21 for the sterilization by the ultraviolet light generator 23, and then flows out from the air outlet 2. The toxic or harmful substances in the air can be inactivated by short-wave ultraviolet light sterilization, which improves the sterilization and disinfection ability of the air sterilizer, and solves the problem of poor sterilization and disinfection effects of the air sterilizer in the prior art.

Optionally, the air sterilizer further includes a reflector 22 located in the first cavity 21. The reflector 22 is provided corresponding to the ultraviolet light generator 23 with its reflective surface facing the UV light generator 23.

Specifically, the reflector 22 may be a flat mirror, and the reflecting surface of the flat mirror is set to face the ultraviolet light generator 23. The reflector 22 can be welded to the inner wall of the housing, or can be fixed to the inner wall of the housing through magnetic adsorption. The shape of the reflector 22 can be a U-shaped plane mirror or a rectangular plane mirror. The shape of the reflector 22 can be set according to actual conditions. The reflector 22 reflects the light emitted by the ultraviolet light generator 23. Such reflections increase the light density and allow the toxic or harmful gases at the same location to be repeatedly irradiated by the ultraviolet light, which increases the sterilization effect of the air sterilizer by multiples.

Optionally, the reflector 22 includes a first sub reflector, a second sub reflector and a third sub reflector, which sub reflectors are sequentially connected, such that the second sub reflector is located between the first sub reflector and the third sub reflector.

Specifically, the first sub reflector, the second sub reflector and the third sub reflector may all be plane mirrors connected in sequence. The first sub reflector and the third sub reflector are located on the same side of the second sub reflector. The angle between the first sub reflector and the second sub reflector may be a right angle or an obtuse angle, and the angle between the third sub reflector and the second sub reflector may also be a right angle or an obtuse angle.

Optionally, the air sterilizer further includes a fan 12, which fan 12 is located in the first cavity 21, and placed between the ultraviolet light generator 23 and the air outlet 2.

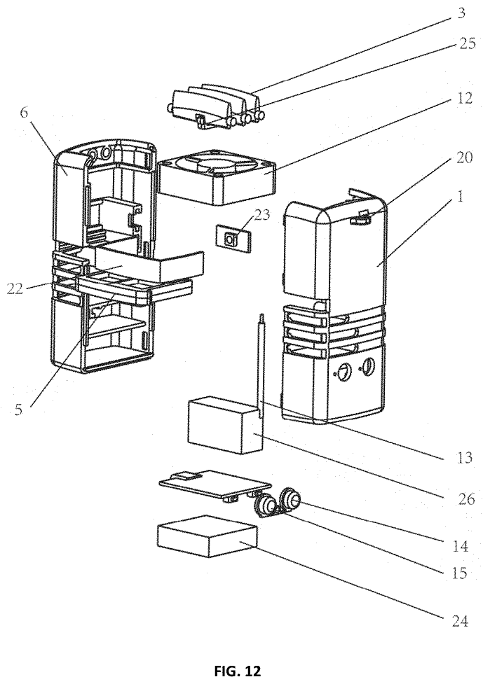

Specifically, as shown in FIG. 12, the fan 12 may be arranged parallel to the top surface of the housing. The fan 12 is connected to a power source, which power source may be a built-in power source of the air sterilizer or a commercial power source. By setting, the air volume output per unit time of the fan 12 can be increased, thereby improving the efficiency of the air sterilizer.

Optionally, the air sterilizer further includes two hanging loops 20 for strap, and such hanging loops 20 are respectively connected to opposite sides of the housing.

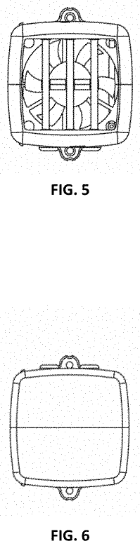

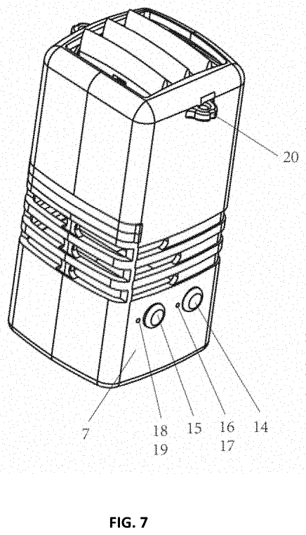

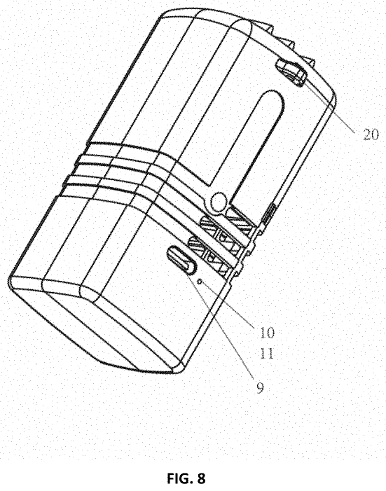

Specifically, as shown in FIGS. 6 and 7, the hanging loops 20 may be located near the top surface of the housing, and are respectively arranged on opposite sides of the housing. The strap can pass through the hanging loops 20, such that the user may hang the air sterilizer around the neck or wear it around the waist, making it convenient for carrying and use.

It should be understood that the size of the air sterilizer can be set according to actual needs. In order to meet the needs of the user to wear it on the body, it can be designed as a small one. Or it can be designed as a large size for indoor use.

Optionally, the air sterilizer further includes a negative ion generator 26 and a cathodic high-voltage carbonized fiber 13. A second cavity is provided in the housing, and the negative ion generator 26 is located in the second cavity. The first end of the cathodic high-voltage carbonized fiber 13 is connected to the negative ion generator 26, while the second end of the cathodic high-voltage carbonized fiber 13 passes through the second cavity and the first cavity 21 in turn and located at the air outlet 2.

Specifically, as shown in FIG. 12, the second end of the cathodic high-voltage carbonized fiber 13 passes through the second cavity and the first cavity 21 in sequence, and is set at the air outlet 2. The negative ion generator 26 generates negative high voltage, which is output to the high-voltage carbonized fiber 13 through wire for tip discharge and ionizing the air to generate negative ions. The negative ions condense with dust particles in the air into larger particles and settle on the ground. That process results in dust removal and odor removal.

It should be understood that the number of high-voltage carbonized fiber 13 can be set according to actual needs. It can be two or three. When the number of high-voltage carbonized fiber 13 is more than one, they may be evenly spaced at the outlet 2.

Optionally, the air sterilizer further includes a filter 5. The filter 5 may be located in the first cavity 21, and placed between the ultraviolet light generator 23 and the air inlet 4.

Specifically, the filter 5 can be any one or a combination of more than one of an activated carbon filter, a formaldehyde removal filter, or a deodorization filter. The shape of the filter 5 can be the same as the shape of the cross section of the housing, and it is placed horizontally in the first cavity 21 and can be connected to the housing by bolting. Through the setting of this filter 5, impurities and toxic or harmful gases in the air can be filtered, and the sterilization effect of the air sterilizer can be further improved.

Optionally, the air sterilizer further includes a power interface 9 arranged on the outside of the housing and a rechargeable battery 24 arranged in the second cavity. The power interface 9, the ultraviolet light generator 23 and the negative ion generator 26 are all electrically connected to the rechargeable battery 24.

Specifically, as shown in FIG. 8, the power interface 9 may be arranged in a preset area outside the housing. The preset area refers to an area where its position is not higher than half the height of the housing. As most indoor sockets are generally installed at a distance of about 0.3 meters from the ground, the power interface 9 is set in a preset area to facilitate connection with indoor sockets. The housing is provided with a first opening 10 beside the power interface 9. A first indicator light 11 electrically connected to a rechargeable battery 24 is provided in the second cavity. The light signal of the first indicator light 11 is sent to the outside through the first opening 10. The first indicator light 11 signifies that the rechargeable battery 24 is in states of charging, low battery, or fully charged by flashing, brightness or displaying different colors. For example: the first indicator light 11 emits red light to indicate that the rechargeable battery 24 is charging, emits green light to indicate that the rechargeable battery 24 is fully charged, and emits flashing red light to indicate that the battery level of the rechargeable battery 24 is low.

The rechargeable battery 24 is electrically connected to the fan 12, the ultraviolet light generator 23 and the negative ion generator 26 through the control circuit board 8. The power interface 9 may be connected to the mains, and may also be electrically connected to a vehicle power supply or a portable power supply for charging the rechargeable battery 24. The rechargeable battery 24 provides electrical energy to the air sterilizer, such that it can be used while carried by the user.

Optionally, the air sterilizer further includes a control device and an indicator light located in the second cavity. The housing is provided with an opening adapted to the indicator light. The control device is electrically connected with the ultraviolet light generator 23, the negative ion generator 26 and the indicator light respectively.

Specifically, as shown in FIG. 1 and FIG. 12, the control device is also electrically connected to a power source for supply of electrical power. The first half of the shell 1 is provided with a control panel area 7. The control device is fixedly connected to the control panel area 7 and is located inside the housing. The control device may be a control circuit board 8. The indicator light is electrically connected to the control device and can be firmly set on the control device. The indicator light can be an LED light (light-emitting diode), which indicates working status of the fan 12, ultraviolet light generator 23, and negative ion generation by flashing, brightness or displaying different colors. For example, if the LED light is on, it means the negative ion generator 26 is on. If the LED light is dim, it means the negative ion generator 26 is off. If the LED light flashes, it means the negative ion generator 26 is malfunctioning.

The control device also includes a mechanical switch, which is arranged on the control circuit board 8 and is electrically connected to it. The mechanical switch can be a button switch or a rotary switch. The button switch refers to a switch that uses a button to connect or disconnect an electrical circuit. The button switch is a simple structure operated with low-voltage that can be controlled manually and reset automatically. Rotary switch is a switch that connects or disconnects an electrical circuit through manual control of a rotary handle. Both the button switch and the rotary switch are applicable to the embodiments of the present application. The housing is provided with an opening corresponding to the mechanical switch, so that the mechanical switch is exposed on the surface of the housing for easy control by the user.

Further, as shown in FIGS. 7 and 12, the mechanical switch includes a function selection switch 14 and a sterilization selection switch 15. The function selection switch 14 is electrically connected to the fan 12, the ultraviolet light generator 23 and the negative ion generator 26 through the control device, and can be used to start or stop the fan 12, the ultraviolet light generator 23 and the negative ion generator 26 simultaneously. The sterilization selection switch 15 is electrically connected to the negative ion generator 26 through the control device, and can be used to start or stop the negative ion generator 26. The control circuit board 8 is provided with indicator lights corresponding to the function selection switch 14 and the sterilization selection switch 15 respectively. A second indicator light 16 and a third indicator light 18 are respectively provided on one side of the function selection switch 14 and the sterilization selection switch 15. The function and working principle of the indicator lights have been described above and will not be repeated here. The housing is provided with a second opening 17 and a third opening 19, which are arranged corresponding to the indicator lights, so that the light information of the indicator lights can be sent out to the outside through the openings.

Optionally, the air sterilizer further includes movable shutters 3 arranged at the air outlet 2.

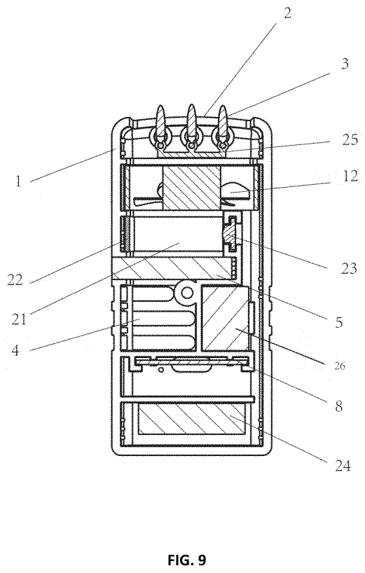

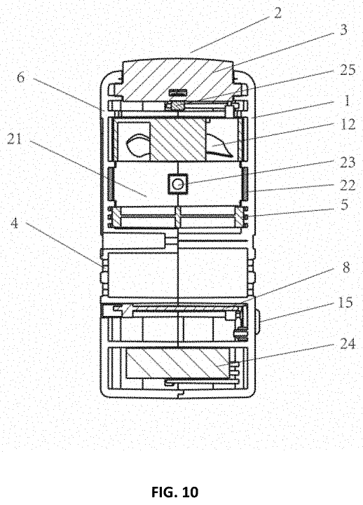

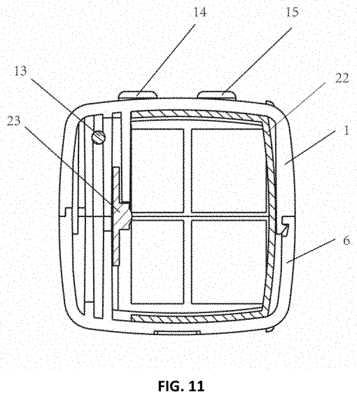

Specifically, as shown in FIGS. 9, 10 and 12, the movable shutters 3 include shutter blades and a movable rod 25. The shutter blades are rotatably connected to the air outlet 2, and mechanically connected to the movable rod 25. Turning the movable rod 25 will cause the shutter blades to rotate. Rotation of the shutter blades will have a guiding effect on the direction of air flowing out of the air outlet 2. Therefore, by adjusting the shutter blades, the air outlet angle of the air sterilizer can be adjusted to meet the diverse needs of users.

The present invention has been described through the above-mentioned technical solutions and examples, but the present invention is not limited to the above-mentioned specific embodiments. The above-mentioned specific embodiments are merely illustrative and not restrictive. Under the enlightenment of the present invention, those of ordinary skill in the art can make many forms of changes without departing from the purpose of the present invention and the protection scope of the claims, all of which fall within the protection of the present invention.

1. An air sterilizer, comprising:
a housing and an ultraviolet light generator,
wherein the housing is provided with a first cavity, an air inlet and an air outlet,
wherein said air inlet and air outlet are located at the first cavity,
wherein the ultraviolet light generator is also located in the first cavity,
wherein the air sterilizer further comprises a reflector located in the first cavity, wherein the reflector is arranged corresponding to the ultraviolet light generator, such that the reflecting surface of the reflector faces the ultraviolet light generator, wherein the reflector comprises a mirror, and
wherein the air sterilizer is portable.

2. (canceled)

3. The air sterilizer according to claim 1, wherein the air sterilizer further comprises a fan, which fan is located in the first cavity, and placed between the ultraviolet light generator and the air outlet.

4. The air sterilizer according to claim 1, wherein the air sterilizer further comprises two hanging loops for strap, and such hanging loops are respectively connected to opposite sides of the housing.

5. (canceled)

6. The air sterilizer according to claim 1, wherein the air sterilizer further comprises a filter, and the filter is located in the first cavity, and placed between the ultraviolet light generator and the air inlet.

7. (canceled)

8. (canceled)

9. The air sterilizer according to claim 1, wherein the reflector includes a first sub reflector, a second sub reflector, and a third sub reflector, which sub reflectors are sequentially connected, such that the second sub reflector is located between the first sub reflector and the third sub reflector.

10. The air sterilizer according to claim 1, wherein the air sterilizer further comprises movable shutters arranged at the air outlet.
